(19) United States
(12) Patent Application Publication
(10) Pub. No.: US 2008/0274061 A1
(43) Pub. Date: Nov. 6, 2008

(54) Method for Treating a Restless Limb Disorder

(76) Inventors: Erwin Schollmayer, Leverkusen (DE); Richard Sachse, Leverkusen (DE); Marina Braun, Dusseldorf (DE)

Correspondence Address: HARNESS, DICKEY, & PIERCE, P.L.C, 7700 Bonhomme, Suite 400, ST. LOUIS, MO, 63105, US

(21) Appl. No.: 12/114,348

(22) Filed: May 2, 2008

Related U.S. Application Data
(60) Provisional application No. 60/915,964, filed on May 4, 2007.

(30) Foreign Application Priority Data
May 4, 2007 (EP) EP 07 009 013.9

(51) Int. Cl. A61K 9/12; A61K 31/381

(52) U.S. Cl. 424/45; 514/438

(57) ABSTRACT

A method for treating a restless limb disorder such as restless legs syndrome in a subject comprises administering, transmucosally in the oronasopharyngeal chamber of the subject, one or more doses of rotigotine or a pharmaceutically acceptable salt, prodrug or metabolite thereof, wherein each such dose comprises an amount effective to reduce occurrence and/or severity of one or more symptoms of the disorder, but wherein the total of all such doses in a 24-hour period does not exceed about 450 μg rotigotine free base equivalent.

### FIELD OF THE INVENTION

This application claims priority under 35 U.S.C. §119 of European Patent Application No. EP 07 009 013.9 filed on May 4, 2007. This application also claims priority of U.S. provisional patent application Ser. No. 60/915,964, filed on May 4, 2007. This application contains subject matter that is related to co-assigned PCT application No. ______, titled “______”, filed concurrently herewith. The disclosure of each of the applications identified in this paragraph is incorporated by reference in its entirety.

The present invention relates to methods for treating restless limb disorders such as restless legs syndrome (RLS), and to pharmaceutical articles, dosage units and pharmaceutical kits useful in practicing such methods.

### BACKGROUND OF THE INVENTION

Restless legs syndrome (RLS) is a neurological disorder that expresses itself as a false sensation in one or both legs accompanied by a strong kinetic urge. Symptoms of RLS include tingling, pulling, aching, itching, burning, cramps or pain, causing in the person affected an irresistible urge to move the affected leg or legs. These symptoms occur most frequently when the person affected is resting or immobile. During extended wakeful sedentary periods, for example while seated in a theater, airplane or automobile, symptoms of RLS can be very troublesome and distressing, and during sleep periods, especially at night, this sensory disorder with its attendant kinetic urge leads to restlessness and disturbed or interrupted sleep. More rarely, similar symptoms can occur in one or both arms.

RLS can occur at any age but becomes progressively more prevalent in older people. In most cases, the severity of the disorder increases with age, but there are exceptions. Some researchers have estimated that RLS affects as many as 12 million people throughout the United States. However, others estimate a much higher occurrence due to underdiagnosis and misdiagnosis.

Silber et al. (2004) Mayo Clin. Proc. 79(7):916-922, for the Medical Advisory Board of the Restless Legs Syndrome Foundation, present an algorithm for management of RLS. In the case of intermittent RLS, defined as RLS that is troublesome enough to require treatment but not frequent enough to necessitate daily therapy, options are said to include non-pharmacological therapy and medications. A non-pharmacological approach is said to involve (1) iron replacement if serum ferritin is low; (2) mental alerting activities; (3) trial of abstinence from caffeine, nicotine and alcohol; and/or (4) consideration of discontinuation of antidepressants, neuroleptic agents, dopamine-blocking antiemetics or sedating antihistamines if this is possible without harming the patient. Medications suggested by the Board include (1) combination carbidopa/levodopa (25 mg carbidopa+100 mg levodopa), optionally in controlled release (CR) form; (2) low-potency opioids such as propoxyphene or codeine, or opioid receptor agonists such as tramadol; (3) benzodiazepines or benzodiazepine receptor agonists such as temazepam, triazolam, zolpidem or zaleplon; and (4) dopamine agonists such as pramipexole or ropinirole.

Carbidopa/levodopa, 25+100 mg (½-1 tablet) can be used for RLS that occurs intermittently in the evening, at bedtime, or on waking during the night, or for RLS associated with specific activities, such as airplane or lengthy car rides or theater attendance. Controlled release carbidopa/levodopa, 25+100 mg (1 tablet) can be used alternatively before bed for RLS that awakens the patient during the night. Even the CR form has a relatively short duration of action and may not produce sustained efficacy if RLS persists throughout much of the night. See Silber et al., id.

Problems with levodopa treatment include augmentation and rebound. Augmentation is defined as a worsening of RLS symptoms earlier in the day after an evening dose of medication, including earlier onset of symptoms, increased intensity of symptoms, or spread of symptoms to the arms. Up to 70% of patients taking levodopa daily will develop augmentation, and the risk increases with daily doses of 200 mg or more. The risk of augmentation may be lower with intermittent use, such as fewer than 3 times per week, but this has not been firmly established. Patients should be warned about augmentation because taking additional doses of levodopa results in worsening augmentation. If augmentation occurs, the drug should be discontinued and another agent substituted. Rebound, defined as recurrence of RLS in the early morning, occurs in 20% to 35% of patients taking levodopa. See Silber et al., id.

Additional information on augmentation and rebound is found in the publications individually cited below.

Earley & Allen (1996) Sleep 19:801-810.

Guilleminault et al. (1993) Neurology 43:445.

Opioids and benzodiazepines are associated with risk of addiction and development of tolerance, and their availability for RLS therapy may therefore be restricted.

Dopamine precursors are drugs that the brain converts to dopamine, a chemical neurotransmitter involved in controlling movement. Dopamine agonists directly stimulate nerves in the brain that are not being stimulated by dopamine. Because of the undesired augmentation and rebound phenomena associated with the dopamine precursor levodopa, many RLS patients now use dopamine agonists. For example, pergolide, pramipexole and ropinirole have been studied for their use in treating RLS and involuntary limb movements. Common side effects associated with dopamine agonists include nausea, congestion, fatigue and fluid retention. See Rowett & Tank (2005) Yahoo! Health Encyclopedia at http://health.yahoo.com/ency/healthwise/ue4948.

Dopamine agonists have the advantage of a longer half-life than levodopa, substantially eliminating concerns about limited duration of effect through a sleep period. The occurrence of augmentation is generally considered to be less with dopamine agonists, and the doses used for treatment of RLS are typically much smaller than the doses used to treat Parkinson's disease. However, since the action of orally administered dopamine agonists generally commences 90 to 120 minutes after ingestion, these agents cannot be used effectively once symptoms have started. See Silber et al. (2004), cited above.

U.S. Patent Application Publication No. 2004/0048779 of Schollmayer proposes a method of treating RLS comprising administering the dopamine agonist rotigotine, for example in the form of a transepicutaneous pharmaceutical preparation such as a patch or plaster. Rotigotine so administered is stated to allow even low dosages to improve a patient's condition without causing intolerable or undesirable effects. Dosage amounts of 0.5 to 10 mg/day are proposed.

U.S. Patent Application Publication No. 2003/0166709 of Rimpler et al. proposes a pharmaceutical composition for parenterally administering N-0923 (rotigotine) in depot form. The composition is stated to be suitable for chronic treatment of diseases such as Parkinson's disease or RLS that are associated with a dopamine metabolic disorder. Although the composition is particularly well suited to administration by injection, it is also stated to be suitable for mucosal, for instance nasal, administration. Suitable daily dosages of 0.5 to 40 mg, ideally 2 to 10 mg, are proposed.

Swart et al. (1995) Pharm. Sci. 1:437-440 reported improved bioavailability of N-0923 (rotigotine) after buccal, nasal or rectal administration to rats in the form of the HCl salt, compared with oral dosing.

International Patent Publication No. WO 2005/063236 of Kramer proposes an intranasal pharmaceutical formulation comprising a pharmaceutically acceptable acid addition salt, e.g., the hydrochloride (HCl) salt, of rotigotine together with a-cyclodextrin. Formulation concentrations of rotigotine HCl of 1 to 6 mg/ml are proposed. Such a formulation is stated to be useful in treatment of Parkinson's disease and other dopamine-related disorders. As background, the '236 publication reports that “[i]t is known that dopamine D2 agonists such as apomorphine or rotigotine may in principle be used to treat morbus Parkinson and other diseases for which an increase in the dopamine level is beneficial such as . . . RLS.”

U.S. Patent Application Publication No. 2001/0053777 of Brecht proposes a method for treating RLS comprising administering an α2 agonist such as clonidine in combination with another neuropsychic drug. Among neuropsychic drugs listed therein are dopamine agonists including N-0923 (rotigotine). It is stated that the combination of drugs can be administered by oral, spinal, anal or intravenous routes, by inhalation, subcutaneously or transdermally.

Rotigotine is under development or already approved in several countries as a transdermal formulation (Neupro® rotigotine patch of Schwarz Pharma). Such a formulation has release properties enabling once daily administration to a subject to provide a relatively stable concentration of rotigotine in plasma of the subject. It would be desirable, however, to have an additional option for rotigotine administration that could supplement the therapeutic benefits of transdermal rotigotine, for example when RLS symptoms become or can be predicted to become particularly acute. It would be especially beneficial if such additional option were capable of providing relatively rapid therapeutic response and/or did not require a very large incremental dosage amount of rotigotine.

More generally, alternative methods for treatment of restless limb disorders such as RLS are needed in the art.

### SUMMARY OF THE INVENTION

There is now provided a method for treating a restless limb disorder such as RLS in a subject, comprising administering, transmucosally in the oronasopharyngeal chamber of the subject, one or more doses of a rotigotine agent, wherein each such dose comprises an amount effective to reduce occurrence and/or severity of one or more symptoms of the disorder, but wherein the total of all such doses in a 24-hour period does not exceed about 450 μg rotigotine free base equivalent.

A “rotigotine agent” herein means rotigotine in any form, e.g., rotigotine in the form of its free base or a pharmaceutically acceptable salt, prodrug or metabolite thereof, or in a combination of such forms.

Illustratively the administration is intranasal.

According to the present invention, a pharmaceutical composition for treatment of a restless limb disorder such as RLS comprises, in a vehicle suitable for oronasopharyngeal, for example intranasal, administration, a rotigotine agent in an amount providing one or more doses, each of about 10 to about 450 μg rotigotine free base equivalent.

A related embodiment of the invention comprises use of a rotigotine agent in an amount providing one or more doses, each of about 10 to about 450 μg rotigotine free base equivalent, in a vehicle suitable for oronasopharyngeal, for example intranasal, administration, in manufacture of a medicament for treatment of a restless limb disorder such as RLS.

There is further provided a pharmaceutical article comprising
(a) a reservoir containing a composition that comprises, in a pharmaceutically acceptable vehicle, a rotigotine agent in an amount providing one or more doses;
(b) indicia, on the reservoir or in or on packaging thereof, for oronasopharyngeal (for example, intranasal) administration of said one or more doses in an amount not exceeding about 450 μg rotigotine free base equivalent per day, for treatment of a restless limb disorder such as RLS; and optionally
(c) a dispenser functionally connected or connectable to the reservoir for dispensing a dosage unit of the composition from the reservoir as an aerosol, atomized spray, liquid drops or insufflatable powder.

There is still further provided a pharmaceutical dosage unit comprising, in a pharmaceutically acceptable vehicle, a rotigotine agent in an amount of about 10 to about 450
g rotigotine free base equivalent. Such a dosage unit is, illustratively, useful in a method of the invention for treating a restless limb disorder such as RLS.

There is still further provided a pharmaceutical kit comprising
(a) a composition that comprises, in a pharmaceutically acceptable vehicle, a rotigotine agent in an amount providing one or more doses; and
(b) a document having indicia for oronasopharyngeal (for example, intranasal) administration of said one or more doses in an amount not exceeding about 450 μg rotigotine free base equivalent per day, in treatment of a restless limb disorder such as RLS.

There is still further provided a pharmaceutical kit comprising
(a) an oral, transdermal or parenteral formulation comprising a first dopamine agonist in an amount effective for chronic treatment of a restless limb disorder; and
(b) an oronasopharyngeal formulation comprising a second dopamine agonist in an amount effective for p.r.n. treatment to reduce occurrence and/or severity of one or more breakthrough symptoms of the disorder;
   wherein the first and second dopamine agonists are the same or different.

There is still further provided a regimen for managing a restless limb disorder in a subject, comprising
(a) administering a first dopamine agonist to the subject by an oral, transdermal, or parenteral route in an amount and frequency effective for chronic treatment of the disorder; and
(b) administering a second dopamine agonist, transmucosally in the oronasopharyngeal chamber of the subject, in an amount effective for p.r.n. treatment to reduce occurrence and/or severity of one or more breakthrough symptoms of the disorder;
   wherein the first and second dopamine agonists are the same or different.

Illustratively, in the above embodiments, at least the second dopamine agonist comprises a rotigotine agent as defined above, and is administered intranasally. Illustratively the first dopamine agonist also comprises a rotigotine agent as defined above, and is administered transdermally.

There is still further provided a method for treating intermittent RLS in a subject, comprising administering a dopamine agonist transmucosally in the oronasopharyngeal chamber of the subject, in an amount effective to reduce occurrence and/or severity of one or more RLS symptoms.

Illustratively, in this embodiment, the dopamine agonist comprises a rotigotine agent as defined above, and is administered intranasally.

### BRIEF DESCRIPTION OF THE DRAWINGS

FIG. 1 provides a graph showing effect of rotigotine treatment on baseline-adjusted RLS symptom severity score over a series of SITs (suggested immobilization tests) as described in Example 7.

FIG. 2 provides a graph showing effect of rotigotine treatment on mean RLS symptom severity scores over time, rated by subjects at 5 minute intervals, as described in Example 7.

FIG. 3 provides a graph showing effect of rotigotine treatment on baseline-adjusted PLMWI (an index of periodic limb movement occurring in sleep) over a series of SITs as described in Example 7.

### DETAILED DESCRIPTION

The present invention is based in part on a finding, in a placebo-controlled clinical trial reported in Example 7 below, that intranasal administration of rotigotine HCl is effective for treatment of RLS at doses much lower than heretofore contemplated for any route of administration, and furthermore that suppression and reduction of symptoms of RLS occurs as soon as 10 minutes after such administration.

A “restless limb disorder” herein is a disorder characterized by an urge, which is often compelling, to move one or more limbs, usually accompanied by and often in direct response to an uncomfortable or unpleasant sensation in the affected limb or limbs. Typically the symptoms are at least partially relieved by movement of the limb or limbs, and are most pronounced during periods of rest or inactivity, including sleep periods and periods of wakeful sedentary immobility. Examples of restless limb disorders include, but are not limited to, RLS and Periodic Leg Movement Disorder (PLMD), which can occur independently but are often present simultaneously in the same subject and can be manifestations of a common underlying cause.

Rados (2006) FDA Consumer Magazine 40(3), http://www.fda.gov/fdac/features/2006/306_rls.html, not admitted to be prior art to the present invention, provides additional information on RLS.

The International RLS Study Group (IRLSSG), in collaboration with the U.S. National Institutes of Health (NIH), has developed four diagnostic criteria for RLS:
(a) a compelling urge to move the legs, usually accompanied or caused by uncomfortable and unpleasant sensations in the legs;
(b) symptoms partially or completely relieved by movement, as long as the movement continues;
(c) onset or worsening of symptoms during periods of rest or inactivity, such as lying or sitting; and
(d) symptoms worsening or occurring only in the evening or at night.

In various embodiments, the restless limb disorder treated by the present method comprises:
(a) a disorder characterized by at least one of the IRLSSG diagnostic criteria;
(b) RLS as characterized by all of the above criteria;
(c) mild, moderate, severe or very severe RLS;
(d) moderate to very severe RLS;
(e) any of the above, accompanied by PLMD; or
(f) PLMD, whether or not accompanied by diagnostic criteria for RLS.

The term “RLS” or “restless legs syndrome” herein is not to be interpreted as necessarily affecting both legs, though most commonly both legs are affected, to similar or differing degrees.

The subject herein can be human or non-human; if non-human, the subject can be an animal, e.g., a mammal, of any species, including domestic animals, farm animals, exotic and zoo animals, laboratory animals, etc. In embodiments of particular interest at present, the subject is a human patient having a restless limb disorder such as RLS, whether clinically diagnosed or not, but most typically meeting the IRLSSG diagnostic criteria.

RLS is generally classified as (1) primary or idiopathic, and (2) secondary. In primary RLS, the cause of the symptoms is not known, or is not associated with any other medical condition known to exist in the patient. Primary RLS has been identified as having a more insidious onset of symptoms, which occur at an earlier age, than in secondary RLS. Patients with primary RLS are more likely to have affected family members than are people in the general population or even those patients with secondary RLS. In secondary RLS, the onset is usually more precipitous and typically occurs after age 40 years. In this case, RLS occurs in relationship to one or more other conditions (e.g., pregnancy, renal failure, neuropathy, diabetes, rheumatoid arthritis, iron deficiency or radiculopathy) or to use of medications (e.g., dopamine receptor antagonists, histamnine-receptor antagonists, selective serotonin reuptake inhibitors and other antidepressant drugs). Symptoms of secondary RLS usually diminish or go away when the associated disease or condition improves or if the implicated medication is stopped.

Because there is currently no laboratory test available to diagnose RLS, a clinical diagnosis is made by evaluating a subject's history and symptoms, for example according to the IRLSSG criteria set forth above.

Based on frequency of symptoms and response to treatment, RLS can be divided into three categories: (1) intermittent or situational; (2) daily; and (3) refractory.

Notwithstanding any definitions of intermittent RLS found in documents cited herein, the term “intermittent RLS” in the present application will be understood, consistent with the definition of Silber et al. (2004), cited above, to mean RLS having symptoms troublesome enough to require treatment but not frequent enough to require daily treatment. Symptoms of intermittent RLS may occur sporadically, periodically (e.g., seasonally or in relation to the menstrual cycle), or may be associated with particular provocative states, such as a sedentary event (e.g., meeting, theater, dinner party, air travel or car travel). Such occurrences of intermittent RLS symptoms are referred to herein as “flares”. Daily RLS is characterized by symptoms of an intensity and frequency sufficient to necessitate daily therapy. Refractory RLS is characterized by a failure to respond to therapy that in most patients is generally adequate.

A scoring system for RLS symptom severity, called the RLS Rating Scale, has been developed by IRLSSG. It is often used in clinical trials and other studies to evaluate therapeutic effects of treatment. The system uses 10 questions, each scored 0-4, with higher scores representing more severe symptoms. Results for all 10 are then added together to give an overall score or diagnostic index, with severity described as mild (overall score of 0 to 10); moderate (overall score of 11-20); severe (overall score of 21-30); and very severe (overall score of 31-40). The patient rates his/her symptoms during the last week in response to the following ten questions:
1. Overall, how would you rate the RLS discomfort in your legs or arms?
   _(4) Very severe
   _(3) Severe
   _(1) Mild
   _(0) None
2. Overall, how would you rate the need to move around because of your RLS symptoms?
   _(4) Very severe
   _(3) Severe
   _(1) Mild
   _(0) None
3. Overall, how much relief of your RLS arm or leg discomfort did you get from moving around?
   _(4) No relief
   _(3) Mild relief
   _(2) Moderate (1 to 3 hours per 24 hour)
   _(1) Either complete or almost complete relief
   _(0) No RLS symptoms to be relieved
4. How severe was your sleep disturbance due to your RLS symptoms?
   _(4) Very severe
   _(3) Severe
   _(1) Mild
   _(0) None
5. How severe was your tiredness or sleepiness during the day due to your RLS symptoms?
   _(4) Very severe
   _(3) Severe
   _(1) Mild
   (0) None
6. How severe was your RLS as a whole?
   _(4) Very severe
   _(3) Severe
   _(1) Mild
   _(0) None
7. How often did you get RLS symptoms?
   _(4) Very often (6 to 7 days in 1 week)
   _(3) Often (4 to 5 days in 1 week)
   _(2) Sometimes (2 to 3 days in 1 week)
   _(1) Occasionally (1 day in 1 week)
   _(0) Never
8. When you had RLS symptoms, how severe were they on average?
   _(4) Very severe (8 hours or more per 24 hour)
   _(3) Severe (3 to 8 hours per 24 hour)
   _(2) Moderate (1 to 3 hours per 24 hour)
   _(1) Mild (less than 1 hour per 24 hour)
   _(0) None
9. Overall, how severe was the impact of your RLS symptoms on your ability to carry out your daily affairs, for example carrying out a satisfactory family, home, social, school or work?
   _(4) Very severe
   _(3) Severe
   _(1) Mild
   _(0) None
10. How severe was your mood disturbance due to your RLS symptoms—for example angry, depressed, sad, anxious or irritable?
   _(4) Very severe
   _(3) Severe
   _(1) Mild
   _(0) None

In some embodiments, diagnosis of RLS in a subject is made based at least in part on a score of ≧11 on the RLS Rating Scale, for example ≧12, ≧13, ≧14 or ≧15.

Periodic limb movement disorder (PLMD), also known as nocturnal myoclonus, is a sleep disorder where the patient moves involuntarily during sleep. PLMD is characterized by leg twitching or jerking movements during sleep that typically occur every 10 to 60 seconds, sometimes throughout the night. The movements range from small shudders of the ankles and toes to kicking and flailing of the arms and legs. Sometimes, oral, nasal and abdominal movements also occur. The periodic jerking often wakes the individual (as well as his or her sleeping partner) and can significantly disturb quality of sleep. PLMD is a cause of insomnia and daytime sleepiness. As with RLS, incidence of this disorder increases with age.

The difference between RLS and PLMD is that PLMD occurs while people are sleeping and has no symptoms, while RLS keeps people awake because of the symptoms. Although most subjects with RLS experience or will develop PLMD, most people with PLMD do not experience RLS. Finally, like RLS, the cause of PLMD is unknown.

The method of the present invention comprises administering a rotigotine agent, for example rotigotine free base or a pharmaceutically acceptable salt, prodrug or metabolite thereof, to a subject. Rotigotine is the INN (international nonproprietary name) for the chemical substance (−)-5,6,7,8-tetrahydro-6-[propyl[2-(2-thienyl)ethyl]amino]-1-naphthol:

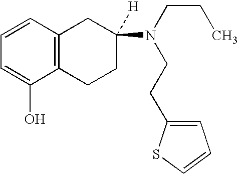

Rotigotine can also be identified as the (S)-enantiomer of 5,6,7,8-tetrahydro-6-[propyl[2-(2-thienyl)ethyl]amino]-1-naphthol. The content of (R)-enantiomer in the rotigotine administered or in a pharmaceutical composition useful herein is low, for example less than about 10 mol %, less than about 2 mol % or less than about 1 mol %, based on the total quantity of rotigotine present.

Because rotigotine free base can be prepared from rotigotine salt, any mention of rotigotine free base herein does not exclude the presence of trace amounts of rotigotine salts, for example, rotigotine HCl. Trace amounts of salt impurities typically do not exceed about 10% by weight, for example, do not exceed about 5%, about 2%, about 1%, or about 0.1% by weight of rotigotine free base. It should further be noted that rotigotine free base can be used in combination with other forms of the compound, for example, the HCl salt, in greater than trace amounts.

In one embodiment, the method comprises administering rotigotine free base. In another, presently preferred, embodiment, the method comprises administering an acid addition salt of rotigotine, for example, rotigotine HCl. Other pharmaceutically acceptable acid addition salts include the oxolinate, tartrate, citrate, phosphate, sulfate and methanesulfonate salts.

In a further embodiment, the compound administered is a prodrug of rotigotine. A prodrug is an agent that generally has weak or no pharmaceutical activity itself but is converted into a pharmaceutically active compound in vivo. Prodrugs are often useful because, in some situations, they may be easier to administer than the corresponding active compound. A prodrug may, for instance, be bioavailable by oral administration where the active compound is not. A prodrug may be simpler to formulate, for example through improved solubility in a pharmaceutical composition, than the active compound.

As a nonlimiting example, prodrugs useful herein can be derivatives of rotigotine at the phenolic hydroxy group thereof, for example, esters (e.g., aryl carbonyl esters, alkyl carbonyl esters or cycloalkyl carbonyl esters, in particular alkyl carbonyl esters and cycloalkyl carbonyl esters each with up to 6 carbon atoms, carbonates, carbamates, acetals, ketals, acyloxyalkyl ethers, phosphates, phosphonates, sulfates, sulfonates, thiocarbonyl esters, oxythiocarbonyl esters, thiocarbamates, ethers and silylethers).

Alkyl carbonyl esters comprise compounds in which the oxygen atom of rotigotine is bonded to a —C(O)-alkyl group. An alkyl carbonyl ester can be formed by esterification of the phenolic hydroxy group of rotigotine with an alkanoic acid, e.g., with acetic acid, propionic acid, butyric acid, isobutyric acid or valeric acid.

Cycloalkyl carbonyl esters comprise compounds in which the oxygen atom of rotigotine is bonded to a —C(O)-cycloalkyl group.

Aryl carbonyl esters comprise compounds in which the oxygen atom of rotigotine is bonded to a —C(O)-aryl group.

Carbonates comprise compounds in which the oxygen atom of rotigotine is bonded to a —C(O)—O—R group, where R is as defined below.

Carbamates comprise compounds in which the oxygen atom of rotigotine is bonded to a —C(O)—NRR¹, —C(O)—NH—R¹ or —C(O)—NH₂ group, where R and R¹ are as defined below.

Acetals comprise compounds in which the oxygen atom of rotigotine is bonded to a —CH(OR)R¹ group, where R and R¹ are as defined below.

Ketals comprise compounds in which the oxygen atom of rotigotine is bonded to a —C(OR)R¹R² group, where R, R¹ and R² are as defined below.

Acyloxyalkyl ethers comprise compounds in which the oxygen atom of rotigotine is bonded to a —CHR—O—C(O)—R¹ or —CH₂—O—C(O)—R¹ group, where R and R¹ are as defined below.

Phosphates comprise compounds in which the oxygen atom of rotigotine is bonded to a —P(O₂H)OR group, where R is as defined below.

Phosphonates comprise compounds in which the oxygen atom of rotigotine is bonded to a —P(O₂H)R group, where R is as defined below.

Sulfates comprise compounds in which the oxygen atom of rotigotine is bonded to a —S(O)₂OR group, where R is as defined below.

Sulfonates comprise compounds in which the oxygen atom of rotigotine is bonded to a —S(O)₂R group, where R is as defined below.

Thiocarbonyl esters comprise compounds in which the oxygen atom of rotigotine is bonded to a —C(═S)—R group, where R is as defined below.

Oxythiocarbonyl esters comprise compounds in which the oxygen atom of rotigotine is bonded to a —C(═S)—O—R group, where R is as defined below.

Thiocarbamates comprise compounds in which the oxygen atom of rotigotine is bonded to a —C(═S)—N—RR¹, —C(═S)—NH—R¹ or —C(═S)—NH₂ group, where R and R¹ are as defined below.

Ethers comprise compounds in which the oxygen atom of rotigotine is bonded to a —R group, where R is as defined below.

In the above examples of prodrugs, each of R, R¹ and R² is independently hydrogen, alkyl (e.g., C₁₋₆ alkyl), cycloalkyl (e.g., C₃₋₁₀ cycloalkyl) or aryl (e.g., phenyl).

In some embodiments, each of R, R¹ and R² is independently C₁₋₆ alkyl, C₃₋₁₀ cycloalkyl or phenyl.

Alkyl groups can be branched or unbranched and typically have 1 to 10 carbon atoms, for example C₁₋₆ alkyl. Alkyl groups can be unsubstituted or substituted with one or more substituents, for example halogen substituents.

Cycloalkyl groups may have only ring-forming C atoms or may optionally bear further C atoms. Illustratively, cycloalkyl groups have 3-10, 4-8 or 4-6 C atoms.

Phenyl groups can optionally be substituted in one or more positions (e.g., with alkoxy, alkyl, halogen and/or nitro substituents).

Illustrative prodrugs of rotigotine are described, for example, in the publications individually cited below.

Den Daas et al. (1990) Naunyn Schiedebergs Arch. Pharmacol. 341:186-191.

Den Daas et al. (1991) J. Pharm. Pharmacol. 43:11-16.

The suitability of a prodrug of rotigotine can, for example, be determined by incubating a particular prodrug candidate under defined conditions with an enzyme cocktail and a cell homogenizate or an enzyme-containing cell fraction, and measuring the active rotigotine. A suitable enzyme mixture is for example the S9 liver preparation distributed by Gentext of Woburn, Mass. Other methods to test the suitability of a prodrug are known to those skilled in the art.

For example, in vitro conversion of a prodrug into the active substance can be assayed in the following way. The microsome fraction containing essential metabolic enzymes is obtained from liver cell homogenizates from humans, monkeys, dogs, rats and/or mice by differential centrifugation; alternatively, it is also possible to obtain the cytoplasmic fraction. The subcellular fraction is suspended with a buffer in such a way that a solution with a defined protein content is obtained. After the addition of a 1 μM solution of the prodrug to be tested, it is incubated at 37° C. for 60 minutes. Then rotigotine is quantified by means of HPLC/UV or HPLC/MS and related to the quantity used. For more detailed analyses, concentration or time series are investigated.

In a further embodiment, the compound administered is a metabolite of rotigotine. An example of such a metabolite of rotigotine is (S)-2-N-propylamino-5-hydroxytetralin, as disclosed for example in International Patent Publication No. WO 2005/058296.

According to the method of the present invention, the rotigotine agent, for example rotigotine HCl, is administered transmucosally in the oronasopharyngeal chamber of the subject. The “oronasopharyngeal” chamber herein is the chamber formed by the interconnected oral, pharyngeal and nasal cavities, each of which have a mucosal lining. The drug is not only administered via this chamber, but is administered “transmucosally” therein, by which is meant that at least a substantial part of the absorption of the drug occurs across the mucosal lining of one or more of the oral, pharyngeal and nasal cavities. This is important, as any portion of the drug that is directed to or swallowed into the gastrointestinal system and absorbed there is subject to first-pass metabolism in the liver, a process that is known to reduce bioavailability of orally (i.e., perorally) administered rotigotine to close to zero. Illustratively, administration is intraoral (e.g., buccal, palatal or sublingual), pharyngeal, intranasal or any combination thereof. In one embodiment the rotigotine agent, for example rotigotine HCl, is administered intranasally.

For transmucosal delivery, the rotigotine agent, for example rotigotine HCl, can be administered as unformulated active pharmaceutical ingredient (API), but for most purposes is more suitably formulated in a pharmaceutical composition suitable for deposition on and retention by the mucosa. Such a composition comprises the rotigotine agent in a pharmaceutically acceptable vehicle, which can be solid (e.g., a powder), semi-solid (e.g., a gel or paste) or liquid (e.g., an aqueous or non-aqueous medium). A composition suitable for transmucosal administration in the oronasopharyngeal chamber is referred to herein as an “oronasopharyngeal composition” or “oronasopharyngeal formulation”; analogously a composition suitable for intraoral administration is referred to as an “intraoral composition” or “intraoral formulation” and one suitable for intranasal administration as an “intranasal composition” or “intranasal formulation”. Nonlimiting examples of oronasopharyngeal compositions useful herein include oral sprays, nasal sprays, nasal drops and insufflatable powders. An “insufflatable” powder is a powder composition having particle size and other properties rendering it suitable for insufflation to a bodily cavity, in the present instance most particularly to the nasal cavity.

In one embodiment, the pharmaceutical composition is adapted for intranasal administration. This means that the composition is in a form physically suitable for intranasal delivery of a therapeutic agent. The composition can be administered, for example, as a nasal spray, nasal drop, suspension, gel, ointment, cream, or powder. The composition can illustratively be administered by propelling particles (e.g., powder particles or atomized liquid droplets) into the nose from a dispenser that applies a propulsive force to the composition; by dropping liquid into the nose under the force of gravity; or by inserting a nasal tampon or a nasal sponge impregnated with the composition. Without being held to a particular theory, it is believed that most of the absorption of the rotigotine agent when administered intranasally is through the nasal mucosa.

In one embodiment, the intranasally deliverable composition is in the form of an insufflatable powder. Such a powder can be prepared by methods known in the art comprising mixing the active agent in solid particulate form with a suitable powder base such as lactose or starch, or adsorbing a liquid preformulation of the active agent on to the powder base.

In another embodiment, the intranasally deliverable composition is in the form of a sprayable liquid (e.g., one having the rotigotine agent dissolved or dispersed in an aqueous medium).

NovaDel Pharma, Inc. recently reported that a ropinirole oral spray is being developed as a potential treatment for Parkinson's disease. See http://www.novadel.com/pdf/NVDfactsheet.pdf.

Where rotigotine doses, amounts and concentrations are expressed herein as rotigotine “free base equivalent” doses, amounts and concentrations, no inference should be made that the rotigotine is necessarily present in free base form. One of skill in the art will recognize, for example, that 1 mg rotigotine free base (MW=315 approx.) is equivalent to about 1.115 mg rotigotine HCl (MW=351.5 approx.).

According to some embodiments, the rotigotine or salt, prodrug or metabolite thereof is present in a sprayable liquid composition for oronasopharyngeal, more particularly intranasal, administration at a rotigotine free base equivalent concentration of at least about 0.1 mg/ml, or at least about 0.5 mg/ml. For example, a rotigotine free base equivalent concentration of about 0.5 to about 5 mg/ml, or about 1 to about 3 mg/ml, will generally be suitable. In one embodiment, rotigotine HCl is present in the composition at a rotigotine HCl salt concentration of about 1.25 to about 2.5 mg/ml.

The composition administered according to these embodiments comprises an aqueous vehicle that optionally comprises one or more pharmaceutically acceptable excipients, for example, ingredients useful as solubility enhancing agents, tonicifying agents, buffering agents (e.g., phosphates or acetates), acidifying agents, viscosity modulating agents, surfactants, preservatives, antioxidants, antimicrobial agents, etc.

When the composition is formulated in an aqueous medium, it can comprise one or more pharmaceutically acceptable tonicifying (tonicity modulating) agents. Tonicifying agents are used to adjust the composition to a desired range of tonicity, typically to provide a substantially isotonic solution. Examples of tonicifying agents include glycerol, mannitol, sorbitol, sodium chloride (saline solution), potassium chloride and other electrolytes, and combinations thereof.

The composition optionally comprises one ore more pharmaceutically acceptable solubility enhancing agents. Cyclodextrins are examples of solubility enhancing agents, and include α-, β- and γ-cyclodextrins. In one embodiment wherein the active agent is rotigotine HCl, the composition comprises a-cyclodextrin in an amount effective to maintain the rotigotine HCl in solution at a desired concentration, for example as set forth above. According to some embodiments, one or more cyclodextrins, for example a-cyclodextrin, are present in the composition at a concentration of about 1 to about 500 mg/ml, for example about 10 to about 100 mg/ml, about 20 to about 80 mg/ml, about 30 to about 70 mg/ml or about 40 to about 60 mg/ml, illustratively about 50 mg/ml.

The composition optionally comprises one or more pharmaceutically acceptable viscosity modulating agents. Illustrative examples of viscosity modulating agents include glycerol and carboxymethylcellulose. The viscosity of a liquid intranasal composition, for example, can suitably be about 0.5 to about 1.5 mm²/s, for example, about 1 to about 1.4 mm²/s, though viscosities outside these ranges can be useful in particular circumstances. In some embodiments, the viscosity modulating agent comprises glycerol and/or carboxymethylcellulose. Viscosities can be measured by any known method; viscosities recited herein are as determined using an Ubbelohde capillary viscosimeter with suspending ball-level according to DIN 51562, part 1.

Glycerol used herein as a tonicifying and/or viscosity modulating agent can have additional benefits. For example, glycerol can have a soothing effect on nasal mucosa. Furthermore, glycerol has been shown to enhance absorption of rotigotine through bovine nasal mucosa in in vitro permeation assays, as reported in above-cited International Patent Publication No. WO 2005/063236.

The pH of an aqueous liquid composition useful herein is desirably about 4.5 to about 6.0, for example about 5.5 to about 6.1. A pH of about 5.8 is believed to provide optimal rotigotine uptake. The pH of the composition can be adjusted during or after its preparation with a pharmaceutically acceptable buffering and/or acidifying agent, for example, acetate and/or phosphate buffer salts and/or citric acid. In some embodiments, an intranasal composition comprising phosphate buffered saline (PBS) is administered.

Intranasally deliverable pharmaceutical compositions useful according to the present invention may be prepared using methods known in the art or as described in above-cited U.S. Patent Application Publication No. 2005/063236.

A method for treating a restless limb disorder such as RLS in a subject comprises, in some embodiments, intranasally administering to the subject a pharmaceutical composition comprising a rotigotine agent, for example rotigotine HCl, as described above.

The rotigotine agent, for example rotigotine HCl, is administered in one or more doses, each comprising a dosage amount effective to reduce occurrence and/or severity of one or more symptoms of the disorder, for example sensory symptoms and/or periodic limb movement.

What constitutes an amount effective to reduce occurrence and/or severity of one or more symptoms of the disorder can vary depending on the restless limb disorder to be treated, the severity of the disorder, body weight and other parameters of the individual subject, time of day, level of activity or inactivity of the subject, other medication (if any) administered to the subject, and other factors, and can be readily established without undue experimentation by a physician or clinician based upon the disclosure herein. Preferably the amount of active agent administered in each dose does not exceed an amount causing an unacceptable degree of adverse side effects.

Typically a safe and effective dosage amount per administration will be found in the range of about 10 to about 450 μg, for example about 25 to about 400 μg, about 50 to about 300 μg or about 100 to about 200 μg rotigotine free base equivalent. These dosage amounts are much lower than have heretofore been proposed in the literature for treatment of RLS.

One or more doses in amounts given above can be administered in a 24-hour period. Most commonly, no more than one such dose will be found necessary, for example administered at bedtime. In some situations, however, where symptoms are especially troublesome or where the subject spends a significant part of his/her wakeful part of the day in a situation of sedentary immobility (e.g., while traveling by automobile, bus, train or airplane, while attending classes or a cultural, entertainment or sporting event, or while working in a sedentary occupation), two or more doses may be necessary in a 24-hour period.

Typically, the daily dosage amount, i.e., the total of all doses administered oronasopharyngeally in a 24-hour period, is not greater than about 450 μg, for example not greater than about 400 μg, about 350 μg, about 300 μg or about 250 μg, rotigotine free base equivalent.

Due to the varying frequency and severity of symptoms associated with restless limb disorders (e.g., intermittent, daily and refractory RLS), a wide variety of therapeutic regimens of the present invention can be used to treat subjects suffering from these disorders. Thus, the rotigotine agent, for example rotigotine HCl, may be administered oronasopharyngeally, for example intranasally, on a regular or on an “as-needed” basis. In either case, the present method is capable of providing rapid or substantially immediate relief of symptoms.

For example, for a subject having intermittent RLS, an intranasal composition may be administered as the primary means of treating symptoms on an “as-needed” basis.

For a subject having daily or refractory RLS, an intranasal composition may be used routinely one or more times during a 24-hour period, again as the primary treatment. Alternatively, for such a subject, an intranasal composition can be administered as a supplement to another mode of RLS therapy, for example oral administration of an orally bioavailable dopamine agonist such as ropinirole or pramipexole, or transdermal or parenteral administration of a non-orally bioavailable dopamine agonist such as rotigotine. In one embodiment, an oronasopharyngeal (e.g., intranasal) composition as described herein is administered p.r.n. (as needed) to supplement chronic therapy comprising administration of rotigotine or a pharmaceutically acceptable salt, prodrug or metabolite thereof by transdermal patch.

Therefore, a maximum dosage amount presented herein on a per day basis should not be construed as requiring administration of an oronasopharyngeal composition each and every day. Furthermore, dosage amounts given for individual oronasopharyngeal doses that are substantially lower than the daily maximum should not be construed as requiring more than one administration per day. Still further, where oronasopharyngeal administration supplements chronic rotigotine administration, for example by transdermal patch, it will be recognized that the daily maximum dosage amount of about 450 μg rotigotine free base equivalent applies only to the oronasopharyngeal administration, not to the total amount of rotigotine administered to the subject per day.

In the case of a liquid oronasopharyngeal composition, a single dosage amount of rotigotine free base equivalent is contained in a particular volume of the composition. The volume to be administered depends on the desired dose of rotigotine free base equivalent and on the concentration of rotigotine free base equivalent in the composition. For an intranasal composition, the volume administered to one or both nostrils should be a practical volume; not so small as to be incapable of administration by any known device, but not so great that a substantial portion of the dose is not retained by the nasal mucosa. For example, with respect to a sprayable formulation intended for administration to a human subject in two aliquots, one to each nostril, a volume of about 10 to about 300 μl, for example about 12.5 to about 200 μl or about 20 to about 100 μl, can suitably be administered to each nostril, for a total of about 20 to about 600 μl per dose, for example about 25 to about 400 μl or about 40 to about 200 μl per dose. It is generally desirable to administer as low a volume as practicable, to reduce any tendency for the composition to be partially lost by drainage through the nasopharyngeal passage. If desired, an entire dose can be administered to one nostril.

In one embodiment, a dosage volume of about 25 to about 250 μl of a pharmaceutical composition provides a rotigotine free base equivalent dose of about 25 to about 450 μg.

Because the symptoms associated with restless limb disorders occur most frequently when a subject is resting, the optimum time for administration is prior to a period of relative immobilization, for example during or within about 4 hours, about 2 hours, about 1 hour, about 30 minutes or about 15 minutes prior to such a period. Except where otherwise indicated herein, the phrase “period of relative immobilization” means a period during which the subject is sitting or lying down for a majority of the time. This includes wakeful and sleep periods, for example, wakeful sedentary periods and sleep periods. The phrase “sleep period” herein means a time during which the subject is abed or otherwise wishes to sleep (e.g., taking a nap), whether or not the subject is actually asleep. The phrase “wakeful sedentary period” herein refers to a time during which the subject is normally awake but not physically active (e.g., going to a theater to watch a movie, sitting in a chair to read a book, traveling as a passenger in a car or airplane, etc.).

RLS has a debilitating effect, not only because of the actual symptoms, but also because of the decreased quality and duration of sleep that often results from these symptoms. Therefore, in some embodiments, the rotigotine agent is administered in a dosage amount effective to enhance duration and/or quality of sleep during a sleep period. The phrase “duration of sleep” is a quantitative measure of the total amount of time a subject sleeps during a sleep period. Duration of sleep can be expressed as a total amount of time or as “sleep efficiency”, which is expressed as the percentage of time a subject sleeps during a sleep period. The phrase “quality of sleep” herein can refer to a subjective assessment given by a subject of how restorative and undisturbed his/her sleep has been, and/or to one or more objective measures. The subjective assessment can be achieved through a standard questionnaire administered to the subject. Objective assessments include polysomnographic recordings, and monitoring of wrist activity movements, head movements and/or eyelid movements.

It is believed, without being bound by theory, that effectiveness of oronasopharyngeal administration of rotigotine or a salt, prodrug or metabolite thereof in reducing occurrence and/or severity of symptoms of a restless limb disorder depends in part on plasma concentrations of rotigotine achieved following such administration. The phrase “maximum level of rotigotine in plasma” herein means the maximum plasma concentration of rotigotine following oronasopharyngeal, for example intranasal, administration, i.e., in pharmacokinetic (PK) terms, the Cₘₐₓ associated with such administration. In one embodiment, the oronasopharyngeal, e.g., intranasal, composition is formulated to deliver rotigotine in a manner effective to provide a maximum level of rotigotine in plasma of a subject within about 4 hours after administration, for example within about 2 hours, within about 1 hour, within about 30 minutes, within about 20 minutes, within about 15 minutes or within about 10 minutes, after administration. It is believed that such delivery can provide onset of therapeutic benefit very quickly (e.g., within about 30 minutes, within about 20 minutes or within about 10 minutes) after administration, making the method very appropriate for p.r.n. treatment.

In one embodiment, an oronasopharyngeal, e.g., intranasal, composition is administered for acute treatment of RLS. The term “acute treatment” according to the present embodiment is defined as treatment when RLS symptoms become, or can be predicted to become, particularly acute, and when providing relatively rapid therapeutic response is therefore very beneficial.

Independently of onset time, it is generally desirable that a therapeutically beneficial effect should be of sufficient duration that administration to the subject can occur with a dosing frequency no greater than about 10 times a day, for example no greater than about 8, about 6, about 4 or about 3 times a day. Most typically, oronasopharyngeal administration according to the present method will occur no more than twice a day, and in many cases no more than once a day. Thus, in some embodiments, the composition is formulated to deliver rotigotine in a manner effective to provide a sufficient level of rotigotine in plasma of the subject to be efficacious in reducing one or more symptoms of a restless limb disorder for a period lasting at least about 1 hour, for example at least about 2 hours, at least about 3 hours, at least about 4 hours, at least about 6 hours, at least about 8 hours or at least about 12 hours.

Thus, illustratively, one embodiment of the invention relates to oronasopharyngeal, e.g., intranasal, administration of a pharmaceutical composition formulated to deliver rotigotine in a dose and manner effective to reduce severity of sensory symptoms of RLS by at least about 1 point, for example at least about 2 points, on a 0-10 severity scale within about 4 hours after administration.

Another illustrative embodiment of the invention relates to oronasopharyngeal, e.g., intranasal, administration of a pharmaceutical composition formulated to deliver rotigotine in a dose and manner effective to reduce severity of sensory symptoms of RLS by at least about 1.5 point, for example at least about 2 points, on a 0-10 severity scale within about 1 hour after administration.

Another illustrative embodiment of the invention relates to oronasopharyngeal, e.g., intranasal, administration of a pharmaceutical composition formulated to deliver rotigotine in a dose and manner effective to reduce severity of sensory symptoms of RLS by at least about 1 point, for example at least about 2 points, on a 0-10 severity scale from a time about 1 hour after administration for a period of at least about 3 hours.

Another illustrative embodiment of the invention relates to oronasopharyngeal, e.g., intranasal, administration of a pharmaceutical composition formulated to deliver rotigotine in a dose and manner effective to effect an improvement in sensory symptoms of RLS on a 0-10 severity scale within about 1 hour, for example within about 30 minutes, within about 20 minutes, within about 15 minutes or within about 10 minutes, after administration.

An example of such a 0-10 severity scale for sensory symptoms of RLS has been used in a clinical trial as described in Example 7 hereof. This scale is not to be confused with the RLS Rating Scale developed by IRLSSG as described above. Other scales can be used, for example a 100-mm visual analogue scale from 0 (no symptoms) to 100 (very severe symptoms), as reported by Stiasny-Kolster et al. (2006) Mov. Disord. 21(9):1333-1339, not admitted to be prior art to the present invention.

Severity of motor symptoms can be measured according to the Periodic Limb Movement during Wakefulness Index (PLMWI), as used for example in the clinical trial described in Example 7 hereof.

Thus another illustrative embodiment of the invention relates to oronasopharyngeal, e.g., intranasal, administration of a pharmaceutical composition formulated to deliver rotigotine in a dose and manner effective to reduce severity of motor symptoms of RLS by at least about 3 PLMWI points, for example by at least about 5, about 10, about 15 or about 20 PLMWI points, within about 4 hours after administration.

Another illustrative embodiment of the invention relates to oronasopharyngeal, e.g., intranasal, administration of a pharmaceutical composition formulated to deliver rotigotine in a dose and manner effective to reduce severity of motor symptoms of RLS by at least about 5 PLMWI points, for example by at least about 10 or at least about 15 PLMWI points, within about 1 hour after administration.

Another illustrative embodiment of the invention relates to oronasopharyngeal, e.g., intranasal, administration of a pharmaceutical composition formulated to deliver rotigotine in a dose and manner effective to reduce severity of motor symptoms of RLS by at least about 5 PLMWI points, for example by at least about 10, about 15 or about 20 PLMWI points, from a time about 1 hour after administration for a period of at least about 3 hours.

Illustrative examples of intranasal formulations that will generally be found suitable are compositions comprising rotigotine HCl in a vehicle that comprises PBS, α-cyclodextrin, glycerol and citric acid.

As one example, an illustrative 2.5 mg/ml rotigotine HCl formulation comprises, or in a more particular embodiment consists essentially of:
rotigotine HCl, 2.5 mg/ml
α-cyclodextrin, 50 mg/ml
glycerol, 31.2 mg/ml
citric acid, q.s. for pH adjustment to approximately 5.8
PBS, q.s. to 1 ml

As a further example, an illustrative 1.25 mg/ml rotigotine HCl formulation comprises, or in a more particular embodiment consists essentially of:
rotigotine HCl, 1.25 mg/ml
α-cyclodextrin, 25 mg/ml
glycerol, 31.2 mg/ml
citric acid, q.s. for pH adjustment to approximately 5.8
PBS, q.s. to 1 ml

In various embodiments, a method of the invention comprises intranasal administration of one of the above formulations, or a formulation that is substantially bioequivalent thereto. A “substantially bioequivalent” formulation in the present context is one that exhibits, upon administration to human subjects in accordance with standard PK principles, a bioavailability (as measured, for example, by PK parameters including Cₘₐₓ and AUC₀₋ₜ) that is about 80% to about 125% of that exhibited by the reference formulation. PK data for a test formulation in comparison with a reference formulation as described above may be determined by those of ordinary skill without undue experimentation by comparative testing in a PK study.

More broadly, a method of one embodiment comprises intranasal administration of a formulation comprising rotigotine HCl and α-cyclodextrin in an aqueous medium, or a formulation substantially bioequivalent thereto.

In all of the above embodiments of the invention, the rotigotine agent can be administered in monotherapy. As used herein, “monotherapy” means a therapeutic method for treatment of a condition or disorder involving administration of only one drug. In the present method, monotherapy for a restless limb disorder can involve oronasopharyngeal administration as described herein, and no other therapy. Where the rotigotine agent is administered oronasopharyngeally in combination with or as a supplement to another dosage form (e.g., transdermal patch or depot injection) of a rotigotine agent, such administration is also considered “monotherapy” herein, even if the forms of rotigotine used are different (e.g., intranasal rotigotine HCl in combination with or as a supplement to transdermal rotigotine free base).

Alternatively, the rotigotine agent can be administered in co-therapy with a second active agent (i.e., an agent other than a rotigotine agent) effective for the treatment of a restless leg disorder or a condition associated therewith. In one embodiment of a co-therapeutic method of the invention, oronasopharyngeal administration of a rotigotine agent is used as a p.r.n. treatment to supplement chronic administration of a different active agent, for treatment of a restless limb disorder such as RLS. (As stated above, where such a p.r.n. treatment supplements chronic treatment with a rotigotine agent, the method is considered a form of monotherapy, not co-therapy.)

A suitable agent other than rotigotine for chronic treatment of RLS can illustratively be selected from the following list:
amantadine
apomorphine
bromocriptine
cabergoline
carmoxirole,
(S)-didesmethylsibutramine
dopexamine
fenoldopam
ibopamine
lergotrile
lisuride
memantine
mesulergine
pergolide
piribedil
pramipexole
quinagolide
ropinirole
roxindole
talipexole

Illustratively in the present embodiment, the agent providing chronic treatment of RLS is a dopamine agonist other than rotigotine, for example pramipexole or ropinirole.

In another embodiment, the rotigotine agent is oronasopharyngeally administered concomitantly with a second active agent that addresses a condition associated with the restless limb disorder (e.g., pregnancy, renal failure, neuropathy, diabetes, rheumatoid arthritis, iron deficiency or radiculopathy). One of skill in the art can readily identify a suitable dosage form, route of administration and dosage amount for any particular second active agent from publicly available information in printed or electronic form, for example on the internet. Illustratively and without limitation, such a second active agent can include one or more anticonvulsants, antidepressants, sleeping agents, opioids or opioid receptor agonists, benzodiazepines or benzodiazepine receptor agonists, iron supplements and combinations thereof. The following illustrative examples include pharmaceutically acceptable salts thereof, and combinations thereof.

A suitable anticonvulsant can illustratively be selected from the following list:
gabapentin
carbamazepine
oxycarbazepine
phenytoin
lacosamide
lamotrigine
zonisimade

A suitable antidepressant can illustratively be selected from the following list:
citalopram
paroxetine
fluoxetine
mirtazapine
trazodone
sertraline

A suitable sedative can illustratively be selected from the following list:
amobarbital
aolpidem
bromazepam
brotizolam
chloral hydrate
chlorpromazine
clonazepam
diphenhydramine
doxylamine succinate
eszopiclone
ethchlorvynol
flunitrazepam
fluphenazine
flurazepam
frifluoperazine
glutethimide
haloperidol
ketamine
lormetazepam
loxapine succinate
midazolam
nitrazepam
oxazepam
pentobarbital
perphenazine
prochlorperazine
ramelteon
temazepam
thiothixene
triazolam
tryptophan
zaleplon
zolpidem
zopiclone

A suitable analgesic or pain reliever can illustratively be selected from the following list:
acetaminnophen
alfentanil
ALGRX-4975
allylprodine
alphaprodine
anileridine
benzylmorphine
bezitramide
buprenorphine
butorphanol
clonitazene
codeine
cyclazocine
desomorphine
dextromoramide
dextropropoxyphene
dezocine
diampromide
diamorphone
dihydrocodeine
dihydromorphine
dimenoxadol
dimepheptanol
dimethylthiambutene
dioxaphetyl butyrate
dipipanone
eptazocine
ethoheptazine
ethylmethylthiambutene
ethylmorphine
etonitazene
fentanyl
heroin
hydrocodone
hydromorphone
hydroxypethidine
isomethadone
ketobemidone
levallorphan
levorphanol tartrate
levophenacyl-morphan
lofentanil
meperidine
meptazinol
metazocine
methadone
metopon
morphine
myrophine
nalbuphine
nalorphine
naloxone
narceine
NCX-701
nicomorphine
NO-naproxen
norlevorphanol
normethadone
normorphine
norpipanone
opium
oxycodone
oxymorphone
papaveretum
pentazocine
phenadoxone
phenazocine
phenomorphan
phenoperidine
piminodine
piritramide
proheptazine
promedol
properidine
propiram
propoxyphene
sufentanil
tilidine
tramadol

A suitable benzodiazepine or benzodiazepine receptor agonist can illustratively be selected from the following list:
clonazepam
triazolam
flunitrazepam
lorazepam
nitrazepam
oxazepam
alprazolam
diazepam

In practice of the method of the present invention, an oronasopharyngeal composition can be administered by any known type of dispenser for such a composition. For example, a liquid composition for administration to oral mucosa can be dispensed by means of an oral spray dispenser. Intranasal compositions may be administered through various types of dispensers (e.g., nebulizer, pressurized container, dry powder inhaler, metered dose inhaler, etc.). A dispenser useful herein may be fimctionally connected or connectable to, or integral with, a reservoir that contains a solution, suspension or powder comprising the active agent.

Thus, in one embodiment, the present invention provides a pharmaceutical article comprising a reservoir containing a composition that comprises, in a pharmaceutically acceptable vehicle, a rotigotine agent in an amount providing one or more doses, each dose comprising for example about 10 to about 450 μg rotigotine free base equivalent. The article of this embodiment further comprises indicia, on the reservoir or in or on packaging thereof, for oronasopharyngeal, e.g., intranasal, administration of one or more such doses in an amount not exceeding about 450 μg rotigotine free base equivalent per day, for treatment of a restless limb disorder, e.g., RLS.

The phrase “indicia for administration” herein means information about, or instructions or directions for, use of a pharmaceutical composition or article in treatment of a condition or disorder. Such information, instructions or directions can include, without limitation, details as to operation of a dispenser, dosage amounts, frequency and time of use, safety precautions, etc., in the context of the treatment indicated (in the present instance a restless limb disorder such as RLS). Indicia for administration can be printed directly on the article, for example on the reservoir, or on a label affixed thereto, or enclosed in or affixed to packaging of the article.

In one embodiment, the reservoir is adapted for functional connection to a dispenser for dispensing a dosage unit of the composition from the reservoir as an aerosol, atomized spray, liquid drops or insufflatable powder. For example, the article according to this embodiment can be a “refill” for a dispenser.

In another embodiment, the article itself further comprises a dispenser functionally connected to or connectable to the reservoir. Included in this embodiment is an article wherein the reservoir and dispenser are essentially integral, as in the case, for example, of a squeezable bottle wherein application of pressure to the sides of the bottle (i.e., reservoir) causes the composition contained within it to be forcibly ejected as spray droplets or powder particles.

Where the article is for administration of a liquid intranasal composition, the dispenser can comprise an atomization device configured for insertion into a nostril, and means for actuating the device to deliver the composition into the nostril.

Any sprayable liquid composition as described above can be delivered by such a device. The reservoir can, if desired, be provided separately from the atomization device and actuating means, in which case it is typically adapted for coupling to the atomization device and actuating means prior to use, for example, immediately prior to use.

The atomization device can be any device capable of generating droplets of liquid composition when the composition is supplied from the reservoir. In one embodiment, the atomization device comprises a nozzle or constricted passage that, when the liquid composition passes through it under pressure, breaks the liquid up into droplets. Any means known in the art for actuating the atomization device can be employed, for example application of pressure as by squeezing the reservoir or depressing a plunger, or in the case of an electrically operated device, activating a switch.

The range of droplet size produced by the device is dependent upon the physical properties of the composition, for example its viscosity, the nature of the atomization device (e.g., size of a nozzle aperture) and the manner in which the device is actuated to discharge the composition. Droplets should generally not be so fine as to form an inhalable aerosol, but not so coarse as to fail to adhere to the oronasopharyngeal mucosa.

Optionally, the device is operable to deliver a metered volume of the composition, for example a volume of about 25 to about 600 μl, for example about 20 to about 400 μl or about 40 to about 200 μl per dose. The device optionally adjusts to deliver different metered volumes corresponding to different dosages (e.g., 50 μl, 100 μl, 150 μl, 200 μl or 250 μl of the composition, corresponding in the case of an illustrative composition having a rotigotine free base equivalent concentration of 1 mg/ml, to rotigotine free base equivalent dosages of 50 μg, 100 μg, 150 μg, 200 μg or 250 μg respectively, per administration.

In a further embodiment, the present invention provides a pharmaceutical dosage unit, for example a metered dosage unit, useful for treatment of a restless limb disorder such as RLS, comprising, in a pharmaceutically acceptable vehicle, a rotigotine agent in an amount of about 10 to about 450 μg rotigotine free base equivalent. The term “dosage unit” herein means a pharmaceutical composition in a unit quantity, generally a unit volume, suitable as a single dose. In one aspect, the dosage unit is packaged individually, for example as a single-dose capsule, cartridge or refill. In another aspect, the dosage unit is the product of a single actuation of a dispenser, for example as described above. Thus a dosage unit can illustratively take the form of an atomized or atomizable liquid, for example having a volume, e.g., a metered volume, of about 25 to about 600 μl.

In a still further embodiment, the prevent invention provides a pharmaceutical kit comprising:
(a) a composition that comprises, in a pharmaceutically acceptable vehicle, a rotigotine agent in an amount providing one or more doses, each dose comprising for example about 10 to about 450 μg rotigotine free base equivalent; and
(b) a document having indicia for oronasopharyngeal, for example intranasal, administration of one or more such doses in an amount not exceeding about 450 μg rotigotine free base equivalent per day, in treatment of a restless limb disorder such as RLS.

The document can be supplied together with or separately from the composition. The term “document”, in the present context, will be understood to take any physical or virtual form, including without limitation a label, brochure, advertisement, prescription, instruction sheet, audio recording, audiovisual recording, CD-ROM, website, etc. The composition can have packaging or a label that references the document.

In a still further embodiment, the prevent invention provides a pharmaceutical kit comprising:
(a) an oral, transdermal or parenteral formulation comprising a first dopamine agonist in an amount effective for chronic treatment of a restless limb disorder; and
(b) an oronasopharyngeal formulation comprising a second dopamine agonist in an amount effective for p.r.n. treatment to reduce occurrence and/or severity of one or more breakthrough symptoms of the disorder;
   wherein the first and second dopamine agonists are the same or different.

“Chronic” treatment herein refers to treatment that continues for an extended period and typically involves regular administration of a transdermal or parenteral formulation not more than twice daily, for example once daily, twice weekly or once weekly. The goal of chronic treatment is to provide a “base” level of the first dopamine agonist in plasma of a subject, that can be “topped up” if breakthrough symptoms occur or at times of maximum need. Such “topping up” can be provided by “p.r.n.” (pro re nata) treatment with the oronasopharyngeal formulation, which can be especially useful, for example, during or prior to a period of relative immobilization such as a sleep period, or when the “base” level is at trough, or when symptoms become particularly severe, distressing or troublesome.

The term “breakthrough symptoms” herein refers to symptoms not adequately controlled, alleviated or prevented by the chronic treatment for any reason. For example, breakthrough symptoms can occur as a result of (a) the subject entering or being in a sleep period or a wakeful sedentary period, (b) a flare of intermittent RLS, (c) relatively low plasma level of the first dopamine agonist, for example at or around trough, (d) other predisposing factors, or any combination of the above.

Illustratively, the first and second dopamine agonists can be independently selected from amantadine, apomorphine, bromocriptine, cabergoline, carmoxirole, (S)-didesmethylsibutramine, dopexamine, fenoldopam, ibopamine, lergotrile, lisuride, memantine, mesulergine, pergolide, piribedil, pramipexole, quinagolide, ropinirole, rotigotine, roxindole, talipexole, pharmaceutically acceptable salts, prodrugs and metabolites thereof, and combinations thereof. In one embodiment the first and second dopamine agonists independently comprise pramipexole, ropinirole or a rotigotine agent, for example rotigotine HCl. Illustratively at least the second dopamine agonist comprises a rotigotine agent. In a particular embodiment, the first dopamine agonist comprises a rotigotine agent, for example rotigotine free base, administered transdermally (e.g., as a patch formulation such as Neupro(I rotigotine patch of Schwarz Pharma), or parenterally (e.g., as a depot injectable formulation), and the second dopamine agonist comprises a rotigotine agent, for example rotigotine HCl, administered intranasally (e.g., as a nasal spray).

In a related embodiment, the present invention further provides a regimen for managing a restless limb disorder such as RLS in a subject. The regimen of this embodiment comprises:
(a) administering a first dopamine agonist to the subject by an oral, transdermal, or parenteral route in an amount effective for chronic treatment of the disorder; and
(b) administering a second dopamine agonist, transmucosally in the oronasopharyngeal chamber of the subject, in an amount effective for p.r.n. treatment to reduce occurrence and/or severity of one or more breakthrough symptoms of the disorder;
   wherein the first and second dopamine agonists are the same or different.

As in the kit embodiment described above, the first and second dopamine agonists can be independently selected from amantadine, apomorphine, bromocriptine, cabergoline, carmoxirole, (S)-didesmethylsibutramine, dopexamine, fenoldopam, ibopamine, lergotrile, lisuride, memantine, mesulergine, pergolide, piribedil, pramipexole, quinagolide, ropinirole, rotigotine, roxindole, talipexole, pharmaceutically acceptable salts, prodrugs and metabolites thereof, and combinations thereof. In one embodiment the first and second dopamine agonists independently comprise pramipexole, ropinirole or a rotigotine agent, for example rotigotine HCl. Illustratively at least the second dopamine agonist comprises a rotigotine agent. In a particular embodiment, the first dopamine agonist comprises a rotigotine agent, for example rotigotine free base, administered transdermally (e.g., as a patch formulation such as Neupro® rotigotine patch of Schwarz Pharma), or parenterally (e.g., as a depot injectable formulation), and the second dopamine agonist comprises a rotigotine agent, for example rotigotine HCl, administered intranasally (e.g., as a nasal spray).

In a still further embodiment, a method is provided for treating intermittent RLS in a subject, comprising administering a dopamine agonist transmucosally in the oronasopharyngeal chamber of the subject, in an amount effective to reduce occurrence and/or severity of one or more RLS symptoms. Such administration can be chronic, but is more typically p.r.n., i.e., on an as-needed basis. In the case of p.r.n. administration, it can constitute essentially the sole treatment for the condition, or can be a component of a regimen, such as that described above, further comprising chronic treatment.

Again, the dopamine agonist can illustratively comprise amantadine, apomorphine, bromocriptine, cabergoline, carmoxirole, (S)-didesmethylsibutramine, dopexamine, fenoldopam, ibopamine, lergotrile, lisuride, memantine, mesulergine, pergolide, piribedil, pramipexole, quinagolide, ropinirole, rotigotine, roxindole, talipexole, or a pharmaceutically acceptable salt, prodrug or metabolite thereof, or a combination thereof. In one embodiment the dopamine agonist comprises pramipexole, ropinirole or a rotigotine agent, for example rotigotine HCl. Illustratively the dopamine agonist comprises a rotigotine agent, for example rotigotine HCl, administered intranasally (e.g., as a nasal spray).

According to any of the above embodiments, the rotigotine agent is administered oronasopharyngeally (e.g., intranasally) in an amount effective to reduce occurrence and/or severity of one or more symptoms of a restless limb disorder such as RLS.

In some embodiments, the one or more symptoms comprise sensory symptoms. In certain embodiments, the composition, kit, method or regimen is effective to reduce the severity of sensory symptoms, for example as measured by a reduction in score on a 0 to 10 scale. Effective methods may result in a reduction of at least about 1 point, for example at least about 2 points, at least about 3 points or at least about 4 points, such reduction being evident within about 4 hours after administration, for example, within about 3 hours, within about 2 hours, within about 1 hour, within about 30 minutes, within about 15 minutes or within about 10 minutes after administration.

In some embodiments, the one or more symptoms comprise motor symptoms. In certain embodiments, the composition, kit, method or regimen is effective to reduce the severity of motor symptoms, for example as measured according to the Periodic Limb Movement during Wakefulness Index (PLMWI). Effective methods may result in a reduction in PLMWI of at least about 3 points, for example at least about 5 points, at least about 7 points or at least about 10, such reduction being evident within about 4 hours after administration, for example, within about 3 hours, within about 2 hours, within about 1 hour, within about 30 minutes or within about 15 minutes after administration.

### EXAMPLES

The following examples are merely illustrative, and do not limit this disclosure in any way.

### Example 1

The following intranasal formulation according to the present invention was prepared:
2.5 g/l rotigotine HCl
85 g/l α-cyclodextrin
8 g/l NaCl
0.2 g/l KCl
1.44 g/l Na₂HPO₄.2H₂O
0.2 g/l KH₂PO₄
31.2 g/l glycerol (87% solution in water)
water to add up to final volume
citric acid for pH adjustment
pH of solution 5.8

Water, 610 ml was adjusted to pH 3 with citric acid and α-cyclodextrin, glycerol and rotigotine HCl were added to give a concentration of 85 mg/ml, 2.6 vol. % and 2.5 mg/ml respectively. Subsequently, 250 ml of 4× PBS buffer solution (having four times the concentration of standard PBS buffer solution, i.e., a concentration of 32 g/l NaCl, 0.8 g/l KCl, 5.76 g/l Na₂HPO₄.2H₂O and 0.8 g/l KH₂PO₄ in water) was added, followed by dropwise addition of 1M citric acid until a pH of 5.8 was reached. Water was used to fill up to a final volume of 1000 ml.

The obtained solution was filtered through 0.22 μm PES filter. The solution may be filled in suitable pharmaceutical containers, e.g., dark vials of 8 ml volume, and is ready for intranasal administration to mammals, including man.

### Example 2

The (maximum) solubility of rotigotine HCl in aqueous solution at room temperature (20° C.) can be significantly improved by use of α-cyclodextrin (α-CD), but there is no significant increase in rotigotine solubility when β-cyclodextrin (β-CD) is used. For cyclodextrin concentrations which are close to the maximum solubility of each of the two cyclodextrin types, 5.03 mg/ml rotigotine HCl could be dissolved in an 0.1 g/ml α-CD solution but only 1.57 mg/ml could be dissolved in a 0.015 g/ml β-CD solution.

The concentration was determined by isocratic HPLC analysis. HPLC column LiChroCART 75×4 mm, Superspher 60 RP-select B 5 μm (Merck), column temperature: 30° C., mobile phase:water/acetonitrile/methanesulfonic acid (65/35/0.05 v/v/v), flow rate: 2 ml/min, injection volume: 50 μl, detection at 220 nm, retention time approx. 1.5 min. The concentration was determined by use of an external reference solution having known concentration.

The results are shown in Table 1.

TABLE 1

| | | |
|---|---|---|
| | Rotigotine HCl | Rotigotine base |
| Cyclodextrin | [mg/ml] | [mg/ml] |

| [g/ml] | α-CD | β-CD | α-CD | β-CD |
|---|---|---|---|---|
| 0 | 1.05 | 1.05 | 0.15 | 0.15 |
| 0.005 | n.d. | 1.34 | n.d. | 0.21 |
| 0.01 | 1.39 | 1.40 | 0.19 | 0.22 |
| 0.015 | n.d. | 1.57 | n.d. | 0.22 |
| 0.05 | 3.0 | * | 0.34 | * |
| 0.1 | 5.03 | * | 0.57 | * |

n.d. = no data available
* = exceeds maximum β-CD solubility in the solution tested

It was found that the solubility of rotigotine HCl is increased five-fold by adding 0.1 g/ml α-CD, whereas the maximum solubility enhancing effect of β-CD was only very moderate (factor 1.6).

Rotigotine base is practically insoluble both in aqueous solution and in aqueous solutions containing α- or β-cyclodextrin.

### Example 3

To evaluate the storage stability of potential intranasal formulations of rotigotine HCl the following formulations were prepared:

Formulation sample A:
2.5 g/l rotigotine HCl
0.5% (v/v) Tween 80
8 g/l NaCl
0.2 g/l KCl
1.44 g/l Na₂HPO₄.2H₂O
0.2 g/l KH₂PO₄
water to add up to final volume
citric acid for pH adjustment, pH 5.8

Water, 470 ml was adjusted to pH 3 with citric acid, and Tween 80 and rotigotine HCl were added to give a concentration of 0.5 vol. % and 2.5 mg/ml respectively. Subsequently, 200 ml of 4× PBS buffer solution was added, followed by dropwise addition of 1M citric acid until a pH of 5.8 was reached. Water was used to fill up to a final volume of 800 ml.

Formulation sample B:
2.5 g/l rotigotine HCl
85 g/l α-cyclodextrin
8 g/l NaCl
0.2 g/l KCl
1.44 g/l Na₂HPO₄.2H₂O
0.2 g/l KH₂PO₄
water to add up to final volume
citric acid for pH adjustment, pH 5.8

Water, 470 ml, was adjusted to pH 3 with citric acid, and α-cyclodextrin and rotigotine HCl were added to give a concentration of 85 mg/ml and 2.5 mg/ml respectively. Subsequently, 200 ml of 4× PBS buffer solution were added, followed by dropwise addition of 1M citric acid until a pH of 5.8 was reached. Water was used to fill up to a final volume of 800 ml.

Stability was determined by measuring the concentration of rotigotine over time using gradient HPLC analysis. HPLC column: Licrospher 100 CN, 5 μm, 125×4.6 mm (Bidhoff), pre column filter: 2 μm, mobile phase A: water/methanesulfonic acid (1000/0.5 (v/v)), mobile phase B: acetonitrile/methanesulfonic acid 1000/0.5 (v/v)), flow rate 1.0 ml/min, profile of the gradient: 0 min 95% A/5% B; 2 min 95% A/5% B; 35 min 40% A/60% B; 38 min 40% A/60% B; 39 min 95% A/5% B; initial pressure approx. 90 bar, injection volume 80 μt, detection at 220 nm and 272 nm, retention time approx. 18 min. All peaks in the chromatogram with an area >0.05% were integrated up to a retention time of 35 minutes to calculate purity of the drug substance. The relative purity is used to calculate the degradation of rotigotine HCl. The results are shown in Table 2.

TABLE 2

| | Sample A | | Sample B | |
|---|---|---|---|---|
| | (Tween 80, | | (α-CD, without | |
| | without α-CD) | | Tween 80) | |
| | 220 nm | 272 nm | 220 nm | 272 nm |
| Rotigotine degradation* | −5.6 | 11.1 | −1.0 | −5.6 |
| 6 weeks 60° C. | | | | |
| Rotigotine degradation* | −6.0 | −9.1 | −0.4 | −2.0 |
| 1 year 40° C. | | | | |
| Rotigotine degradation* | −2.6 | −3.1 | ±0.0 | +0.2** |
| 1 year 25° C. | | | | |

*These values reflect the loss in rotigotine absorption between the start values and the actual test points at the given conditions.
**The apparent increase in purity can be explained by the measurement accuracy of the analytical method. The result should be interpreted as no significant change in purity relative to the starting value at t = 0.

It is readily apparent from Table 2 that α-cyclodextrin (sample B) markedly increased stability of rotigotine HCl as compared to the Tween 80 formulation (sample A). The stabilizing effect of α-cyclodextrin becomes also apparent from a comparative test in an aqueous rotigotine HCl solution. Following storage at 60° C. for 8 weeks, a rotigotine solution of 1.6 mg/ml with α-cyclodextrin showed a decrease in the rotigotine concentration of −0.07 mg/ml, whilst a solution of 1.9 mg/ml of rotigotine without α-cyclodextrin showed a decrease of −0.22 mg/ml.

### Example 4

2.5 g/l rotigotine HCl
50 g/l α-cyclodextrin
4 g/l NaCl
0.1 g/l KCl
0.72 g/l Na₂HPO₄.2H₂O
0.1 g/l KH₂PO₄
31.2 g/l glycerol (87% solution in water)

Water, 470 ml, was adjusted to pH 3 with citric acid, and α-cyclodextrin, glycerol and rotigotine HCl were added to give a concentration of 50 mg/ml and 2.5 mg/ml, respectively.

Subsequently, 200 ml of 2× PBS buffer solution were added, followed by dropwise addition of 1M citric acid until a pH of 5.8 was reached. Water was used to fill up to a final volume of 800 ml.

### Example 5

2.5 mg/ml rotigotine HCl
50 mg/ml α-cyclodextrin
PBS 0.5× (NaCl, KCl, Na₂HPO₄, KH₂PO₄)
31.2 mg/ml glycerol
citric acid (for pH adjustment to approximately 5.8)

### Example 6

1.25 mg/ml rotigotine HCl
25 mg/ml α-cyclodextrin
PBS 0.5× (NaCl, KCl, Na₂HPO₄, KH₂PO₄)
31.2 mg/ml glycerol
citric acid (for pH adjustment to approximately 5.8)

### Example 7

This example relates to a randomized, double-blind, placebo-controlled parallel-group proof-of-concept trial which assessed the efficacy, safety and tolerability of ascending doses of rotigotine nasal spray for the treatment of acute symptoms of RLS in subjects with idiopathic RLS. This was evaluated by subject rating of severity of symptoms (sensory symptoms) on a numeric symptoms severity scale and by the Periodic Limb Movement (PLM) index (motor symptoms) as measured by actigraphy following triggering of symptoms by immobilization.

Notice of this trial was first posted on Oct. 17, 2006 at http://www.clinicaltrials.gov/ct/show/NCT00389831?order=1.

### Subjects

A total of 59 subjects with idiopathic RLS, as confirmed by the RLS Rating Scale, were enrolled at two trial sites. Subjects were included if they were between 18 and 65 years of age, met the diagnosis of idiopathic RLS based on the four cardinal features according to IRLSSG, had an RLS diagnostic index (RLS-DI) of ≧11 points at the eligibility assessment (EA) to confirm the diagnosis of RLS and had daily RLS symptoms at least during the last four weeks prior to EA.

The RLS-DI is a tool used to confirm or exclude a diagnosis of RLS. It provides a diagnostic algorithm which determines the probability of a RLS diagnosis. See Benes et al. (2005) Sleep Medicine 6(Suppl.2):S156. The RLS-DI has 10 items which comprise the essential criteria (5 items), and sleep disturbances, family history of RLS, polysomnographic findings, response to dopaminergic treatment, and neurological expert diagnoses to identify RLS or to exclude other causes of RLS symptoms. To increase specificity, the RLS-DI gives negative weights to items if a cardinal RLS symptom is not present. To be eligible for participation in this trial, each subject had to have an RLS-DI score ≧11.

In addition subjects needed to be L-dopa responders and had to be under L-dopa therapy for at least during the last four weeks prior to EA. A total of 44 subjects were randomized of which 42 were treated at least with one application of rotigotine or placebo nasal spray during the trial. Table 3 summarizes the subject disposition during the trial. In the Safety Set (N=42), the mean age was 53.4 years (±7.5) and ranged from 37 to 65 years, with 95% of the subjects younger than 65 years old. The majority of subjects (64%) were female. All subjects were of Caucasian origin. Overall, there were no important differences between treatment groups at baseline.

TABLE 3
Subject disposition

| | “Placebo | “Rotigotine |
|---|---|---|
| | Group”, n | Group”, n |
| Randomized and treated | 10 | 32 |
| Completed treatment | 10 | 28 |
| Prematurely discontinued from treatment | 0 | 4 |
| (reasons for discontinuation below) | | |
| Lack of efficacy | 0 | 2 |
| Other reasons | 0 | 2 |

Trial medication was administered as ready-to-use nasal spray delivering either 55 or 110 μl rotigotine solution of the 3 dosages or placebo:
(a) 62 μg rotigotine (delivered by 55 μl of 1.25 mg/ml rotigotine HCl)
(b) 124 μg rotigotine (delivered by 110 μl of 1.25 mg/ml rotigotine HCl)
(c) 247 μg rotigotine (delivered by 110 μl of 2.5 mg/ml rotigotine HCl)

The corresponding placebo solutions were matched in volume and appearance. Subjects were treated on 4 consecutive days.

Subjects were randomized in a ratio of 3:1 (rotigotine:placebo) to 1 of the 2 treatment arms. To allow for an intra-individual comparison, all subjects within the “rotigotine group” received placebo treatment on either Day 1 or Day 2 in a randomized order (ratio 1:1) and applied a single delivery of the lowest dose of rotigotine nasal spray (62 μg) or matching placebo. On Day 3 and Day 4 subjects received a single delivery of rotigotine nasal spray in ascending doses of 124 and 247 μg. Subjects randomized to the “placebo group” received a single delivery of matching placebo nasal spray on each treatment day.

The treatment with escalating doses was only continued in a particular subject, if the investigator judged the previous day's dose to be safe and well tolerated.

As shown in Table 4, comparable results were noted when comparing the “placebo group” and the “rotigotine group” of subjects, and for both rotigotine treatment sequences, for time since onset and diagnosis of RLS and other diagnostic criteria. The mean RLS-DI was approximately 16 for both treatment groups indicating a definite diagnosis of RLS (Benes et al. (2005), cited above).

TABLE 4
Diagnosis of RLS: mean (standard deviation)

| | | Rotigotine Group | | |
|---|---|---|---|---|
| | Placebo | Placebo- | Rotigotine- | Total |
| | Group | Rotigotine | Placebo | Rotigotine |
| Variable | N = 10 | N = 17 | N = 15 | N = 32 |
| Time since | 14.3 (14.18) | 14.3 (6.19) | 12.0 (8.02) | 13.3 (7.08) |
| onset of RLS | | | | |
| (years) | | | | |
| Time since | 5.4 (2.53) | 4.9 (2.70) | 6.0 (4.30) | 5.4 (3.53) |
| RLS diagnosis | | | | |
| (years) | | | | |
| Time since | 4.9 (2.08) | 4.9 (2.77) | 5.5 (4.45) | 5.1 (3.61) |
| start of drug | | | | |
| therapy for | | | | |
| RLS (years) | | | | |
| Time since | 3.1 (2.92) | 3.5 (3.24) | 4.3 (3.39) | 3.9 (3.29) |
| start of L-dopa | | | | |
| treatment | | | | |
| (years) | | | | |
| RLS | 16.3 (1.83) | 16.1 (1.05) | 15.9 (1.87) | 16.0 (1.47) |
| Diagnostic | | | | |
| Index | | | | |

Suggested Immobilization Tests (SITs), during which subjects should not voluntarily move, were performed in order to trigger motor and sensory symptoms in the legs. It has been shown that immobilization results in worsening of both motor and sensory components of RLS symptoms.

In order to characterize the subject population observed in this trial by the severity of RLS symptoms at the time of enrollment into the trial, subjects were asked to complete the IRLSSG questionnaire to establish a score on the RLS Rating Scale (IRLS) prior to the start of the first SIT on Day 1.

The IRLS evaluated 10 items including discomfort in arms and legs due to RLS, urge to move, and frequency of symptoms (Allen et al. (2003) Sleep Med. 4:101-119). Each item was scored by the subject on a scale of 0 (not present) to 4 (very severe). The sum score ranges from 0 (no RLS symptoms present) to 40 (maximum severity in all symptoms).

The following ranges are used to determine severity categories:
0=none
1 to 10=mild
11 to 20=moderate
21 to 30=severe
31 to 40=very severe

As shown in Table 5, comparable results in the IRLS sum score were noted when comparing the placebo group and the rotigotine group. The mean IRLS score of 25.8 indicates severe RLS symptoms, with all subjects exhibiting at least moderate symptoms (minimum score of 12).

TABLE 5
IRLS sum scores

| | | Rotigotine Group | | |
|---|---|---|---|---|
| | Placebo | Placebo- | Rotigotine- | Total |
| | Group | Rotigotine | Placebo | Rotigotine |
| Variable | N = 10 | N = 17 | N = 15 | N = 32 |
| n | 10 | 17 | 15 | 32 |
| Mean (SD) | 24.6 (5.40) | 27.5 (5.84) | 24.6 (7.02) | 26.2 (6.49) |
| Median | 23.0 | 29.0 | 25.0 | 27.0 |
| Min | 16.0 | 12.0 | 12.0 | 12.0 |
| Max | 36.0 | 34.0 | 36.0 | 36.0 |

SITs and breaks (to allow for release of symptoms during the post dose treatment) were scheduled as shown in Table 6, wherein intake of trial medication occurred at 0 minutes.

TABLE 6
SIT and break schedule

| | | | Duration of |
|---|---|---|---|
| SIT | Time point (min) | SIT duration | following break |
| SIT-0 (pre-dose) | −30-0 | 30 min maximum | — |
| SIT-1 (post-dose) | 0-40 | 40 min | 10 min |
| SIT-2 (post-dose) | 50-80 | 30 min | 10 min |
| SIT-3 (post-dose) | 90-120 | 30 min | 10 min |
| SIT-4 (post-dose) | 130-160 | 30 min | 10 min |
| SIT-5 (post-dose) | 170-200 | 30 min | 10 min |
| SIT-6 (post-dose) | 210-240 | 30 min | — |

On each treatment day, the trial medication was administered 30 minutes after the start of the pre-dose SIT-0 or as soon as the subject's severity score reaches 5 on the numeric symptom severity scale, whichever is sooner. The last scoring prior to dosing is defined as the baseline value for each individual subject.

### Primary Efficacy Endpoints

1. Severity of RLS symptoms in the legs (sensory symptoms): Assessed by subject ratings on severity of symptoms at the start of each pre-dose and post-dose Suggested Immobilization Test (SIT-0 to SIT-6) and every 5 minutes during each SIT, using a numeric symptoms severity scale.
2. PLM during Wakefulness Index (PLMWI) for each SIT and for specified time sections of each SIT (PLM during awake epochs/h, motor symptoms) was evaluated based on PLM measurements by means of actigraphy, recorded over the entire duration of the SIT period (pre dose and post dose SIT-0 to SIT-6, approximately 4.5 hours).

RLS is usually associated with involuntary PLM occurring in wakefulness (PMLW) and sleep (PMLS) in 80-85% of RLS patients. PLMWI indicates the frequency of PLMW and the degree of motor symptoms during wakefulness and as such supports the assessment of severity of symptoms in addition to subjective ratings of symptoms. A central reader was used to standardize PLM evaluation of the actigraphy measurements performed during the SIT period.

### Results

Severity of symptoms in the legs was assessed by subject ratings using a numeric symptoms severity scale at the start of each pre-dose and post-dose SIT-0 to SIT-6 and every 5 minutes during each SIT. Scores on the scale range from 0 (‘no symptoms’) to 10 (‘very severe’).

Immediately prior to the administration of the nasal spray, the severity scores for each treatment day in the “rotigotine group” were as follows: placebo, 3.4 points; rotigotine 62 μg, 3.2 points; rotigotine 124 μg, 3.6 points; and rotigotine 247 μg, 3.4 points (mean of last subject scores of SIT-0, baseline).

The maximum mean reduction from baseline in severity scores on each treatment day in the “rotigotine group” were as follows: placebo, −0.9 points; rotigotine 62 μg, −0.8 points; rotigotine 124 μg, −2.0 points; and rotigotine 247 μg, −2.3 points.

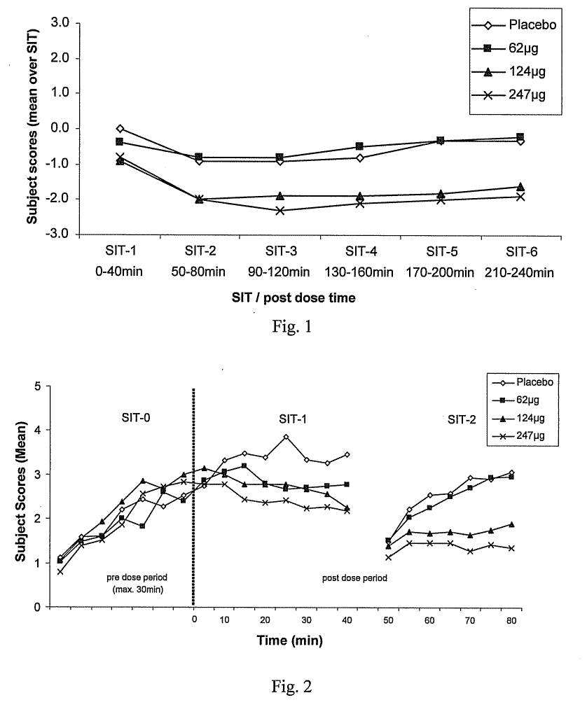

FIG. 1 graphically displays the effect of the nasal spray on average baseline adjusted severity scores per SIT over the post-treatment period (corrected by the individual last pre-dose score).

The effect of the lowest rotigotine dose on the average severity of symptoms per SIT seems to be similar to the effect observed following placebo treatment. A dose dependent effect was noted after administration of the 2 higher rotigotine doses of 124 μg and 247 μg. In general, the effect observed following these doses was a decrease of approximately 2 points in the mean severity scores. This effect is visible already in the average value of SIT-1 (0-40 min. post dose) and remains stable from SIT-2 through SIT-6 until the end of the 4 hour test period.

The mean severity scores over time for the “placebo group” (data not shown) were consistent with the mean severity scores over time for subjects in the “rotigotine group” following placebo treatment, which supports the validity of the observed treatment response (see above). In particular, in the “placebo group” severity scores over time were similar on all treatment days (i.e., there was no “learning effect” during the 4-day treatment period).

In order to evaluate the timing of the onset of action, FIG. 2 graphically displays the course of the mean severity scores over time, rated by the subjects every 5 minutes.

A trend for dose separation across treatments in the severity scores was observed as soon as 10 min. after administration. At this time point, the severity of symptoms triggered by immobilization still increased for placebo and the lowest rotigotine dose, whilst it tended to improve for the two higher doses. It is therefore concluded that onset of action for the two higher rotigotine doses of 124 μg and 247 μg occurred 10 minutes after administration.

As expected, the 10 min. break between SIT-1 and SIT-2 led to a decrease of symptoms resulting in low severity scores at the start of SIT-2. Triggered by immobilization, severity of symptoms increased during SIT-2, which is pronounced in subjects treated with placebo or 62 μg rotigotine, but only mild in subjects treated with 124 μg or 247 μg rotigotine.

Overall, administration of rotigotine nasal spray in doses of 124 μg and 247 μg led to a dose dependent improvement of symptom severity, which was visible 10 min. after administration.

From SIT-2 through SIT-6 this effect was stable until the end of the 4 hours test period. A slight improvement of symptom severity was seen 20 minutes after administration of 62 μg rotigotine nasal spray, which was stable for the remainder of SIT-1 but could not be confirmed during the subsequent SIT periods. However, based on these effects it cannot be excluded that even the lowest dose administered holds some potential for improvement of RLS symptoms.

### PLMW

Frequency of PLMW was obtained from actigraphy measurements performed during the entire SIT period. The PLMWI is defined as the number of PLMWs per hour. Prior to the administration of the nasal spray, the mean PLMWI for each treatment day in the “rotigotine group” was as follows: placebo, 14.3; rotigotine 62 μg, 7.9; rotigotine 124 μg, 16.8; and rotigotine 247 μg, 25.8 (mean PLMWI of SIT-0, baseline).

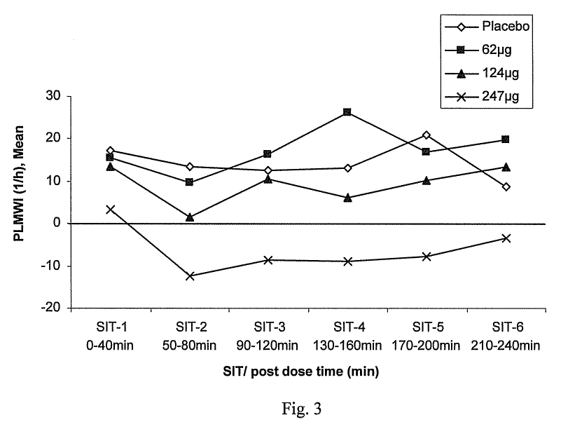

FIG. 3 graphically displays the effect of the nasal spray on the baseline adjusted average PLMWI per SIT (corrected by mean of pre-dose SIT-0) over the post dose period.

An improvement in the PLMWI was observed when subjects were treated with the highest rotigotine dose (247 μg). In general, the effect observed following this treatment was a decrease from baseline of approximately 10 points in the PLMWI. The results of the two lower doses (62 μg and 124 μg) were similar to placebo.

Overall, results of the PLMWI should be interpreted with caution due to the high variability of this parameter.

### CONCLUSIONS

The following conclusions can be drawn:
(a) Administration of rotigotine nasal spray in doses of 124 μg and 247 μg led to a dose-dependent improvement of sensory symptoms.
(b) Onset of action was observed 10 minutes after administration. From SIT-2 through SIT-6 the observed effect was stable until the end of the 4 hours test period.
(c) Based on the effects observed in the severity scores during SIT-1, it cannot be excluded that even the lowest dose (62 μg) administered holds some potential for improvement of RLS symptoms.

What is claimed is:

1. A method for treating a restless limb disorder in a subject, comprising administering, transmucosally in the oronasopharyngeal chamber of the subject, one or more doses of rotigotine or a pharmaceutically acceptable salt, prodrug or metabolite thereof, wherein each such dose comprises an amount effective to reduce occurrence and/or severity of one or more symptoms of the disorder, but wherein the total of all such doses in a 24-hour period does not exceed about 450 μg rotigotine free base equivalent.

2. The method of claim 1, wherein the rotigotine or salt, prodrug or metabolite thereof is administered intranasally.

3. The method of claim 1, wherein the rotigotine or salt, prodrug or metabolite thereof is administered in monotherapy.

4. The method of claim 1, wherein the rotigotine or salt, prodrug or metabolite thereof is administered in co-therapy with another active agent for treatment of the disorder or a condition associated therewith.

5. The method of claim 1, wherein the subject is human.

6. The method of claim 5, wherein the disorder comprises restless legs syndrome (RLS).

7. The method of claim 6, wherein the dose of rotigotine or salt, prodrug or metabolite thereof is effective to reduce severity of sensory symptoms of RLS by at least about 1 point on a 0 to 10 scale within a period of about 4 hours after administration.

8. The method of claim 6, wherein the dose of rotigotine or salt, prodrug or metabolite thereof is effective to reduce severity of sensory symptoms of RLS by at least about 2 points on a 0 to 10 scale within a period of about 1 hour after administration.

9. The method of claim 6, wherein the dose of rotigotine or salt, prodrug or metabolite thereof is effective to effect improvement in sensory symptoms of RLS on a 0 to 10 scale within a period of about 20 minutes after administration.

10. The method of claim 6, wherein the dose of rotigotine or salt, prodrug or metabolite thereof is effective to reduce severity of motor symptoms of RLS by at least about 3 PLMWI points within a period of about 4 hours after administration.

11. The method of claim 6, wherein the dose of rotigotine or salt, prodrug or metabolite thereof is effective to reduce severity of motor symptoms of RLS by at least about 10 PLMWI points within a period of about 1 hour after administration.

12. The method of claim 5, wherein the disorder comprises periodic limb movement disorder (PLMD).

13. The method of claim 5, wherein the dosage amount is about 10 to about 450 μg rotigotine free base equivalent per administration.

14. The method of claim 5, wherein the dosage amount is about 100 to about 450 μg rotigotine free base equivalent per administration.

15. The method of claim 1, wherein the rotigotine or salt, prodrug or metabolite thereof is administered during or within about 2 hours prior to a period of relative immobilization of a limb or limbs affected by the disorder.

16. The method of claim 15, wherein the disorder is RLS and the rotigotine or salt, prodrug or metabolite thereof is administered during or within about 2 hours prior to a wakeful sedentary period or a sleep period.

17. The method of claim 1, wherein the rotigotine or salt, prodrug or metabolite thereof is administered as an active ingredient of an oronasopharyngeally deliverable pharmaceutical composition.

18. The method of claim 17, wherein the composition is formulated to deliver rotigotine in a manner effective to provide a maximum level of rotigotine in plasma of the subject within about 2 hours after administration.

19. The method of claim 17, wherein the composition is formulated to deliver rotigotine in a manner effective to provide a sufficient level of rotigotine in plasma of the subject to be efficacious in reducing one or more symptoms of the disorder for a period lasting at least about 2 hours.

20. The method of claim 17, wherein the composition is a sprayable liquid or insufflatable powder and is administered intranasally.

21. The method of claim 20, wherein the composition is administered in a metered dose from a nasal applicator.

22. The method of claim 21, wherein the composition is a sprayable liquid and the applicator comprises an atomization device.

23. The method of claim 17, wherein the composition comprises an acid addition salt of rotigotine in solution in an aqueous medium.

24. The method of claim 23, wherein the salt is rotigotine hydrochloride.

25. The method of claim 23, wherein the concentration of rotigotine free base equivalent in the composition is about 0.5 to about 5 mg/ml.

26. The method of claim 23, wherein a dosage volume of about 25 to about 250 μl of the composition provides a rotigotine free base equivalent dose of about 25 to about 450 μg.

27. The method of claim 23, wherein the aqueous medium further comprises a pharmaceutically acceptable solubility enhancing agent for the rotigotine salt.

28. The method of claim 27, wherein the solubility enhancing agent comprises α-cyclodextrin.

29. The method of claim 28, wherein the concentration of α-cyclodextrin in the composition is about 10 to about 100 mg/ml.

30. The method of claim 23, wherein the administration is intranasal and the composition so administered is one that (a) comprises, in an aqueous medium, an acid addition salt of rotigotine in a rotigotine free base equivalent concentration of about 0.5 to about 5 mg/ml, and α-cyclodextrin in a concentration of about 10 to about 100 mg/ml; or (b) is substantially bioequivalent thereto.

31. The method of claim 23, wherein the administration is intranasal and the composition so administered is one that (a) consists essentially of
rotigotine HCl, 2.5 mg/ml;
α-cyclodextrin, 50 mg/ml;
glycerol, 31.2 mg/ml;
citric acid, q.s. for pH adjustment to approximately 5.8; and
PBS, q.s. to 1 ml;
or (b) is substantially bioequivalent thereto.

32. The method of claim 23, wherein the administration is intranasal and the composition so administered is one that (a) consists essentially of
rotigotine HCl, 1.25 mg/ml;
α-cyclodextrin, 25 mg/ml;
glycerol, 31.2 mg/ml;
citric acid, q.s. for pH adjustment to approximately 5.8; and
PBS, q.s. to 1 ml;
or (b) is substantially bioequivalent thereto.

33. A pharmaceutical article comprising (a) a reservoir containing a composition that comprises, in a pharmaceutically acceptable vehicle, rotigotine or a pharmaceutically acceptable salt, prodrug or metabolite thereof in an amount providing one or more doses; and (b) indicia, on the reservoir or in or on packaging thereof, for oronasopharyngeal administration of said one or more doses in an amount not exceeding about 450 μg rotigotine free base equivalent per day, for treatment of a restless limb disorder.

34. The article of claim 33, wherein the composition is formulated for intranasal administration.

35. The article of claim 33, wherein the reservoir is adapted for functional connection to a dispenser for dispensing a dosage unit of the composition from the reservoir as an aerosol, atomized spray, liquid drops or insufflatable powder.

36. The article of claim 33, further comprising a dispenser functionally connected or connectable to the reservoir for dispensing a dosage unit of the composition from the reservoir as an aerosol, atomized spray, liquid drops or insufflatable powder.

37. The article of claim 36, wherein the dispenser is adjusted or adjustable to deliver a metered dose.

38. A pharmaceutical dosage unit comprising, in a pharmaceutically acceptable vehicle, rotigotine or a pharmaceutically acceptable salt, prodrug or metabolite thereof in an amount of about 10 to about 450 μg rotigotine free base equivalent in an atomized oronasopharyngeally deliverable composition.

39. A pharmaceutical dosage unit comprising, in a pharmaceutically acceptable vehicle, rotigotine or a pharmaceutically acceptable salt, prodrug or metabolite thereof in an amount of about 10 to about 450 μg rotigotine free base equivalent, wherein the dosage unit has a pH of about 4.5 to about 6.0.

40. A pharmaceutical dosage unit comprising, in a pharmaceutically acceptable vehicle, rotigotine or a pharmaceutically acceptable salt, prodrug or metabolite thereof in an amount of about 10 to about 450 μg rotigotine free base equivalent, wherein the dosage unit is in a metered amount of about 40 to about 200 μl.

41. A pharmaceutical dosage unit comprising, in a pharmaceutically acceptable vehicle, rotigotine or a pharmaceutically acceptable salt, prodrug or metabolite thereof in an amount of about 10 to about 450 μg rotigotine free base equivalent, wherein the dosage unit has a viscosity of about 0.5 to about 1.5 mm²/s.

42. A pharmaceutical kit comprising (a) a composition that comprises, in a pharmaceutically acceptable vehicle, rotigotine or a pharmaceutically acceptable salt, prodrug or metabolite thereof in an amount providing one or more doses; and (b) a document having indicia for oronasopharyngeal administration of said one or more doses in an amount not exceeding about 450 μg rotigotine free base equivalent per day, in treatment of a restless limb disorder.

43. A pharmaceutical kit comprising (a) an oral, transdermal or parenteral formulation comprising a first dopamine agonist in an amount effective for chronic treatment of a restless limb disorder; and (b) an oronasopharyngeal formulation comprising a second dopamine agonist in an amount effective for p.r.n. treatment to reduce occurrence and/or severity of one or more breakthrough symptoms of the disorder; wherein the first and second dopamine agonists are the same or different.

44. The kit of claim 42, wherein at least the second dopamine agonist comprises rotigotine or a pharmaceutically acceptable salt, prodrug or metabolite thereof.

45. The kit of claim 42, wherein the first dopamine agonist comprises rotigotine or a pharmaceutically acceptable salt, prodrug or metabolite thereof, formulated for transdermal or parenteral administration, and the second dopamine agonist comprises rotigotine or a pharmaceutically acceptable salt, prodrug or metabolite thereof, formulated for intranasal administration.

46. A regimen for managing a restless limb disorder in a subject, comprising (a) administering a first dopamine agonist to the subject by an oral, transdermal or parenteral route in an amount effective for chronic treatment of the disorder; and (b) administering, transmucosally in the oronasopharyngeal chamber of the subject, a second dopamine agonist in an amount effective for p.r.n. treatment to reduce occurrence and/or severity of one or more breakthrough symptoms of the disorder; wherein the first and second dopamine agonists are the same or different.

47. The regimen of claim 45, wherein at least the second dopamine agonist comprises rotigotine or a pharmaceutically acceptable salt, prodrug or metabolite thereof.

48. The regimen of claim 45, wherein the first dopamine agonist comprises rotigotine or a pharmaceutically acceptable salt, prodrug or metabolite thereof and is administered transdermally or parenterally, and the second dopamine agonist comprises rotigotine or a pharmaceutically acceptable salt, prodrug or metabolite thereof and is administered intranasally.

49. A method for treating intermittent RLS in a subject, comprising administering a dopamine agonist transmucosally in the oronasopharyngeal chamber of the subject, in an amount effective to reduce occurrence and/or severity of one or more RLS symptoms.

50. The method of claim 48, wherein the dopamine agonist comprises amantadine, apomorphine, bromocriptine, cabergoline, carmoxirole, (S)-didesmethylsibutramine, dopexamine, fenoldopam, ibopamine, lergotrile, lisuride, memantine, mesulergine, pergolide, piribedil, pramipexole, quinagolide, ropinirole, rotigotine, roxindole, talipexole, or a pharmaceutically acceptable salt, prodrug or metabolite thereof, or a combination thereof.

51. The method of claim 48, wherein the dopamine agonist comprises rotigotine or a pharmaceutically acceptable salt, prodrug or metabolite thereof.

52. The method of claim 48, wherein the dopamine agonist is administered intranasally.
